(19) United States
(12) Patent Application Publication
(10) Pub. No.: US 2020/0185109 A1
(43) Pub. Date: Jun. 11, 2020

(54) SYSTEM FOR PROVIDING AN INTEGRATED PLATFORM TO AUTOMATICALLY MANAGE AND IMPROVE PATIENT COMPLIANCE TO TREATMENT AND METHODS OF USE THEREOF

(71) Applicant: Repnets Ltd. Dba Flaskdata.io, Modiin (IL)

(72) Inventor: Danny Lieberman, Modiin (IL)

(21) Appl. No.: 16/608,258

(22) PCT Filed: Oct. 22, 2018
(86) PCT No.: PCT/US18/56946

§ 371 Date: Oct. 25, 2019

Related U.S. Application Data
(60) Provisional application No. 62/575,451, filed on Oct. 22, 2017.

(51) Int. Cl. G16H 50/70; G16H 20/00; G16H 10/60; G16H 40/67

(52) U.S. Cl. CPC G16H 50/70; G16H 40/67; G16H 10/60; G16H 20/00

(57) ABSTRACT

Disclosed is a system and method to provide an integrated platform to automatically manage and improve patient compliance in clinical practice guidelines and protocols over a network. The system includes a common entity store and a plurality of software agents. The common entity store hosts a plurality of clinical trial data provider tenants, a tenant instance per medical indication, historical clinical trial data study, and data pertaining to a plurality of patients. Four agents interact with the common entity store to determine a plurality of actions by monitoring and reinforcing the patient compliance based on the current and historical clinical trial data. The training agent learns from the historical clinical trial data and further creates an expected reward distribution for selecting the plurality of actions from the historical clinical trial data which suggests a highest expected reward. The actions are functions of the measures such as age, gender, and concomitant medication. The RL control agent proposes compliance reinforcement message to the patient agent and the study monitor agent monitors performance measures to create a continuous process of measurement and reinforcement.

### TECHNICAL FIELD

The presently disclosed embodiments are related, in general, to treatment compliance and an automation platform, and specifically relates to an integrated platform to automate protocol selection, monitoring compliance, management, and continuously improving patient compliance to clinical practice guidelines and relevant protocols over a network.

### BACKGROUND

Clinical trials are research studies that explore whether a medical strategy, treatment, or device is safe and effective for humans. These studies also may show which medical approaches work best for certain illnesses or groups of people. When a product is cleared by a regulatory agency such as FDA for marketing, the supporting evidence obtained from clinical trials becomes the basis for clinical practice guidelines, including a dosage of medication or proper intended use of a medical device. Clinical trials are essentially scientific experiments that are monitored in order to assure the safety of subjects, compliance with the study protocol and collection of accurate data. There is a myriad of stakeholders involved in conducting a successful clinical trial process from life science companies, clinical investigators, investigative sites, study monitors, human subjects, nurses etc.

However, there are significant problems with monitoring patient compliance that are related to, or include, slow detection of deviations, slow (or incorrect) response after detection of deviations, and general lack of reuse of knowledge and tools.

In this respect, the monitoring of patients' compliance with the study protocol is particularly crucial because low compliance means more patients need to be recruited in order to achieve the required statistical power for the study. In paper-based clinical trials, response to deviations may take 2-4 months and in trials using electronic data collection, monitoring operations may respond to deviations in 5-8 weeks or more. Slow detection and response during the study push out the resolution of issues to the end of the trial resulting in an end-of-study “data cleaning” process which can take 6 months, delaying the statistical analysis, subsequent regulatory submission, and release of innovative treatment to market. Further, slow detection and response to deviations increase costs. When a patient who is non-compliant is terminated out of a study, the time and money spent on the subject are lost and additional time and money is required to recruit a replacement.

There is currently no systematic way to create reusable knowledge and tools for improving patient compliance in clinical trials. This carries over into medical practice in the challenge for transfer of evidence from randomized controlled clinical trials into medical practice.

In real-life usage, existing strategies for improving the patient compliance to treatment rely on personal data collection and tracking in order to improve the physician-patient interactions so that the physician can monitor the patient for side-effects, and improvement/degradation in the condition.

In spite of important advances in medical therapy in many indications such as cardiovascular disease, derived from better physio-pathological understanding, hospital readmissions rate continue to increase. One possible explanation may be that heretofore known strategies for improving patient compliance via education and physician follow-up do not work. Furthermore, there is a tendency for existing mobile medical applications that collect data and track patient performance to rely either on small and unvalidated datasets of user data acquired online or large, noisy unvalidated datasets of electronic health records.

Consequently, there is a dire need for a system and method that provides an integrated platform to manage and improve patient compliance in clinical trials over a network in order to increase accessibility to study stakeholders and speed up detection and response in order to save time and costs. There is a further need for a system that enables automated monitoring of clinical trial protocol compliance based on clinical measures over/under the threshold as a function of time. Furthermore, there is also a need for a system that learns and formalizes which actions are important in patient compliance for a particular indication from controlled clinical trial data for the benefit of better patient compliance in both clinical trials and in real-life usage.

Further limitations and disadvantages of conventional and traditional approaches will become apparent to one of skill in the art, through comparison of described systems with some aspects of the present disclosure, as set forth in the remainder of the present application and with reference to the drawings.

Czobor, Pál, and Phil Skolnick. “The Secrets of a Successful Clinical Trial: Compliance, Compliance, and Compliance.” Molecular Interventions 11.2 (2011): 107-110. PMC. Web. 22 Oct. 2018.

Shor, et al., “PLATFORM, DEVICE AND METHOD FOR SOCIAL MEDICATION MANAGEMENT” United States Patent Publication 20150235004, published Aug. 20, 2015.

Mazumder et al., Best Subset Selection via a Modern Optimization Lens, first submission in June 2014, arXiv:1507.03133v1 Sat, 11 Jul. 2015 18:19:27 GMT Hanina et al., US Patent Publication US20120316897A1purports to disclose a method and apparatus for monitoring medication adherence. The method includes the steps of determining a present adherence state of a patient, receiving video analysis information reporting on a medication administration session, and determining a next adherence state of a patient based upon the present adherence state of the patient and the video analysis information.

### SUMMARY

The present invention solves many of the problems noted hereinabove as well as others not specifically discussed herein of known patient compliance monitoring systems. In response to the above problems, the present system provides an integrated platform to automatically manage and improve patient compliance in clinical trials over a network. The system includes a common entity store and a plurality of computing device implemented software agents, including a training agent, a study monitor agent, a patient agent, and a reinforcement learning (RL) control agent.

The training agent learns from examples of compliance and non-compliance in historical clinical trial data obtained from the common entity store.

The study monitor agent enables a study monitor to define measures and thresholds, receive alerts and graph trends.

The RL control agent uses reinforcement learning algorithms to provide behavioral reinforcement directly to the patient without relying on physician involvement thereby reducing physician workload and enabling physicians to manage higher-risk exceptions and delegate ongoing compliance to an automated application, caregivers and low-cost phone coaches as applicable.

The patient agent receives messages determined by the RL control agent. The study monitor agent, control and patient agents function cooperatively to create a continuous process of monitoring and reinforcement.

The common entity store hosts a plurality of clinical trial data provider tenants, a tenant instance per medical indication, historical clinical trial data study, and data pertaining to a plurality of patients.

The study monitor agent, RL control agent, training agent, and patient agent function cooperatively to create a continuous process of monitoring, patient compliance measurement and compliance reinforcement, as well as continuous reevaluation of compliance models. The agents all interact with the common entity store to determine a plurality of actions, based on the current and historical clinical trial data, and by monitoring patient compliance and reinforcing or correcting patient's behavior using messaging.

In an aspect of an exemplary embodiment, the training agent learns from the historical clinical trial data what expected rewards have previously been used to reinforce compliance. Furthermore, the training agent is configured to independently suggest or create an expected reward distribution, selecting a plurality of actions which it has detected from historical clinical trial data that suggest to the training agent a highest-value expected reward. The plurality of actions include functions of measures such as age, gender, and concomitant medication.

In one described exemplary embodiment of the invention, the system (sometimes also referred to herein as “Flask Data”) communicates directly with the patients and suggest actions which it has gleaned using reinforcement learning that maximizes a reward of better health in order to achieve a goal of adherence. Initially, the system trains on historical clinical trial data and then the training is reinforced by the system's experience interacting with the patient, i.e. learned knowledge. Alternatively, the system can do a cold-start training, in which the system starts with little or no historical clinical data and only a skeletal protocol and sponsor input. In this embodiment the agents-configured computing devices gather actual clinical data, monitor messaging between the sponsor and subject patients, and the control agent configured computing device assembles a diustribution of what it concludes are the optimal rewards or incentives to obtaining improved patient compliance, on-the-fly, finally sending positive or negative reinforcement (feedback) to the subject in an automated manner.

Accordingly, one advantage of the present invention is that it automates monitoring of a clinical trial protocol compliance enabling delivery to a study sponsor of monitoring as a service (hereinafter “MaaS”) over a widely distributed network and makes high-quality monitoring accessible to any sized project.

Another aspect of exemplary embodiments of the system and methods of the present invention is that it provides actual or nearly-actual real-time detection of deviations as measures which are over or under configured thresholds, as well as responses thereto, since it relies on automated detection instead of manual processing.

Another aspect of exemplary embodiments of the system and methods of the present invention is that data is collected in real-time from patient devices and the system thereby provides MaaS with almost zero-latency detection.

Still another aspect of exemplary embodiments of the system and methods of the present invention is that it is deliverable as an online cloud subscription enabling monitoring and compliance projects of any size to enjoy low-latency detection and response of deviations.

Another aspect of exemplary embodiments of the system and methods of the present invention is that it enables online issue resolution, and in real-time, minimizing the need for cumbersome manual review and clean-up at the end of the study and enabling the delivery of data to study statisticians in days instead of weeks.

Another aspect of exemplary embodiments of the system and methods of the present invention is that it uses automation to increase the return on investment of monitoring budgets.

Another aspect of exemplary embodiments of the system and methods of the present invention is that it reuses historical, controlled clinical data for the benefit of better patient compliance in both clinical trials and real-life usage.

Another aspect of exemplary embodiments of the system and methods of the present invention is that it provides behavioral reinforcement directly to the patient without relying on physician involvement. The practical effects of reducing physician workload includes enabling physicians to delegate ongoing compliance to an automated app and low-cost phone coaches where applicable and to focus on managing higher-risk exceptions/deviations.

Another aspect of exemplary embodiments of the system and methods of the present invention is that the training agent accelerates the learning process by identifying parameters which are truly measures of patient compliance for a particular indication based on controlled clinical trial data, sometimes revealing compliance reward incentives that were not anticipated or appreciated by clinicians or study designers.

Another aspect of exemplary embodiments of the system and methods of the present invention is that it learns even from the relatively small amounts of demonstrations of patient compliance (and non-compliance) in controlled human clinical trials.

These and other features and advantages of the present disclosure may be appreciated from a review of the following detailed description of the present disclosure, along with the accompanying figures in which like reference numerals refer to like parts throughout.

### BRIEF DESCRIPTION OF DRAWINGS

The accompanying drawings illustrate various embodiments of the system, methods of use thereof, and other aspects of the disclosed invention. A person of ordinary skill in the art will appreciate that the illustrated element boundaries (e.g., boxes, groups of boxes, or other shapes) in the figures are merely exemplary. In some examples, one element may be designed as multiple elements, or multiple elements may be designed as one element, without departing meaningfully from the scope of the invention as claimed. In some examples, an element shown as an internal component of one element may be implemented as an external component in another and vice versa, without departing from the scope of the invention as claimed. Further, the elements may not be drawn to scale.

Various embodiments will hereinafter be described in accordance with the appended drawings, which are provided to illustrate and not to limit the scope in any manner, wherein similar designations denote similar elements, and in which:

FIG. 1 illustrates a block diagram of the technical infrastructure of the present system to provide an integrated platform to automatically manage and improve patient compliance in clinical practice guidelines and protocols over a network, in accordance with at least one embodiment;

FIG. 1B is a diagram showing the data and query flows and relationships between the various components of the system;

FIG. 1C is a diagram illustrating the “in-study” role and functioning of the network 108-deployed main system API 109;

FIG. 2 is a diagram detailing the flow of data between the main system API 109 and the common entity store 102;

FIG. 3 is a diagram detailing the flow of data between the network connected device 107 configured with patient agent 103 and the main system API 109;

FIG. 4 is a diagram showing the implementation of a monitor agent according to one exemplary embodiment of the present inventive techniques;

FIG. 5 is a diagram showing the implementation of a training agent according to one exemplary embodiment of the present inventive techniques;

FIG. 6 is a diagram showing the implementation of a control agent according to one exemplary embodiment of the present inventive techniques; and

FIGS. 7A, 7B and 7C are diagrams of the privacy and data isolation features which are part of the configuration of the common entity store.

### DETAILED DESCRIPTION OF EXEMPLARY EMBODIMENTS

The present disclosure is best understood with reference to the detailed figures and description set forth herein. Various embodiments are discussed below with reference to the Figures. However, those skilled in the art will readily appreciate that the detailed description given herein with respect to the figures are simply for explanatory purposes as the methods and systems may extend beyond the described embodiments. For example, the teachings presented and the needs of a particular application may yield multiple alternative and suitable approaches to implement the functionality of any detail described herein. Therefore, any approach may extend beyond the particular implementation choices in the following embodiments described and shown.

References to “one embodiment,” “at least one embodiment,” “an embodiment,” “one example,” “an example,” “for example,” and so on indicate that the embodiment(s) or example(s) may include a particular feature, structure, characteristic, property, element, or limitation but that not every embodiment or example necessarily include that particular feature, structure, characteristic, property, element, or limitation. Further, repeated use of the phrase “in an embodiment” does not necessarily refer to the same embodiment.

The present invention provides a system 100 for automated monitoring and reinforcement of clinical trial protocol compliance based on clinical measures over/under the threshold as a function of time. By examining controlled and cleaned or curated clinical data, the present system learns which actions are important in assessing patient compliance for a particular indication for the benefit of better patient compliance in clinical trials and in real-life usage.

Further, the techniques of the present invention provide behavioral reinforcement directly to the patient, for example encouraging feedback or by suggesting actions that maximize an expected reward for compliance, thereby enabling physicians to delegate ongoing compliance to the automated techniques of the present invention and low-cost phone coaches where applicable.

Exemplary embodiments of the inventive techniques may be realized in hardware or a combination of hardware and software. The inventive techniques may be deployed and configured in a centralized fashion, in at least one specialized computer system, or in a distributed fashion, where different elements may be spread across several interconnected appropriately configured computer systems. Network connected devices (“NCD”) may include computing devices and network connected medical devices which may be configured for executing the techniques described herein. A combination of hardware and software may be a general-purpose computer system with a computer program that, when loaded and executed, may control the computer system such that it carries out the methods described herein. The present disclosure may be realized in hardware that comprises a portion of an integrated circuit that also performs other functions.

Three user interfaces are provided for the three general kinds of users intended to interact and interface with system 100: subject patients, clinical trial or study sponsors, and the system owner.

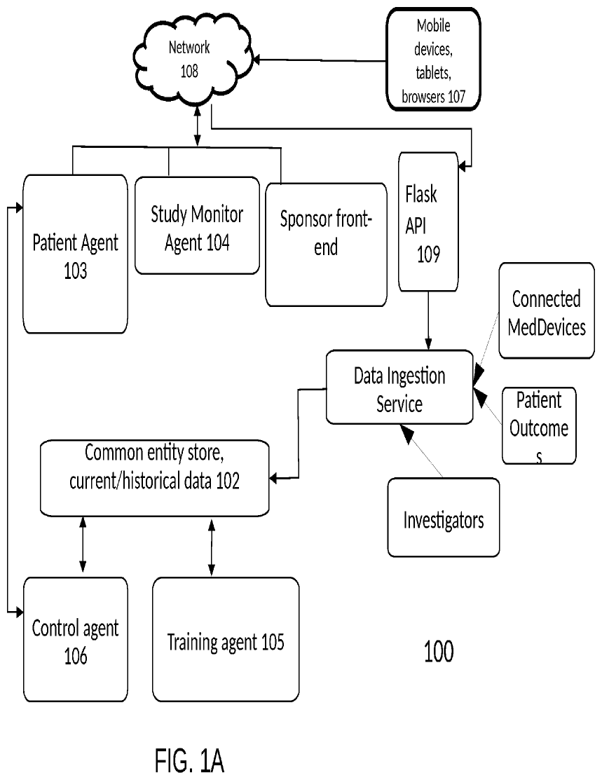

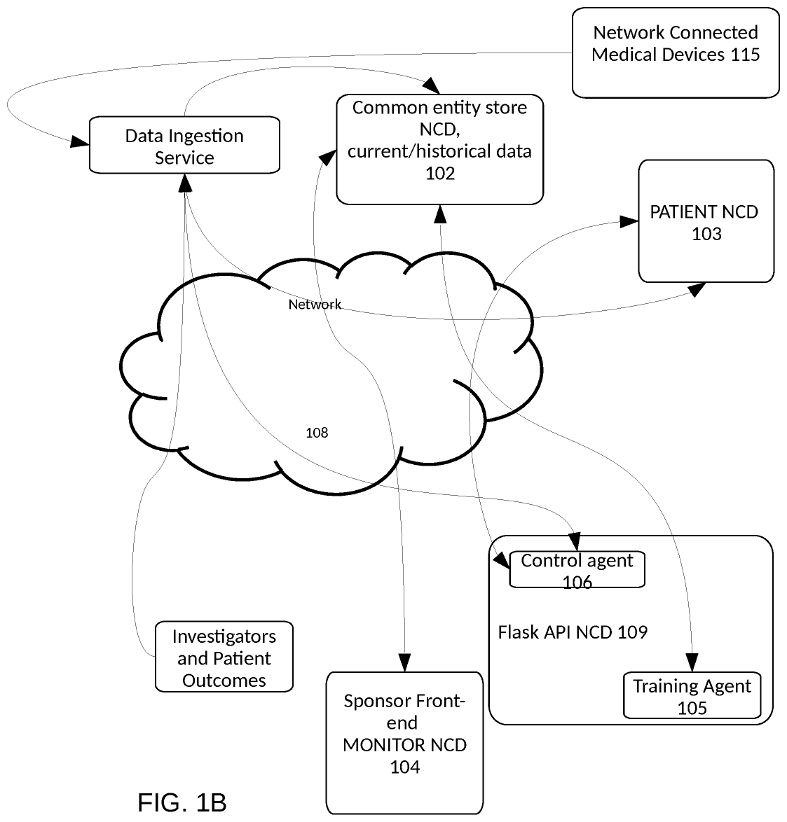

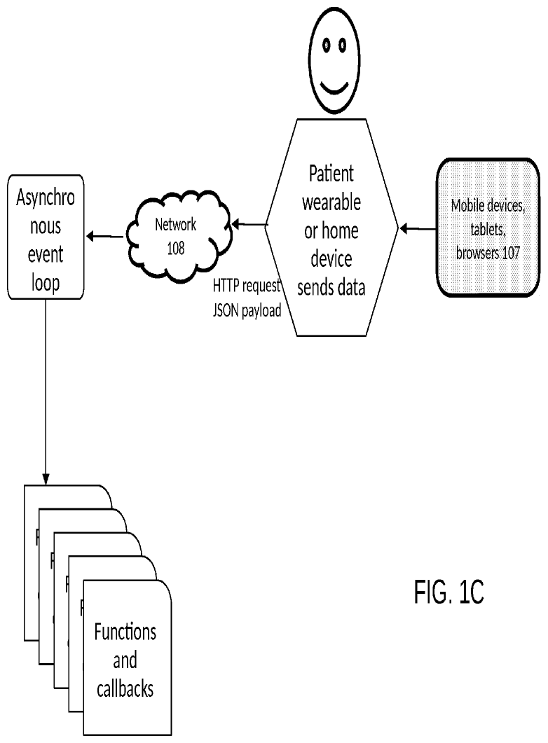

FIG. 1 illustrates the main components of the technical infrastructure of the present system 100 to provide an integrated platform to automatically manage and improve patient compliance in clinical practice guidelines and protocols over a network 108, in accordance with at least one embodiment. The network 108 connects a common entity store 102 and a plurality of software agents 103,104,105, and 106 implemented on a plurality of network connected devices including computing units and medical devices. The plurality of software agents includes a training agent 105, a study monitor agent 104, a patient agent 103, and a reinforcement learning (RL) control agent 106. It will be understood that the main system API 109 may be accessed by patient agents 103 and study monitor agents 104 deployed on multiple user devices and medical devices through one or more computing units 107, or applications residing on the computing units 107. Examples of the computing unit 107 include, but are not limited to, a smartphone, a personal computer, a laptop, a personal digital assistant (PDA), a mobile device, a tablet, or iPad and a medical device with Internet connectivity.

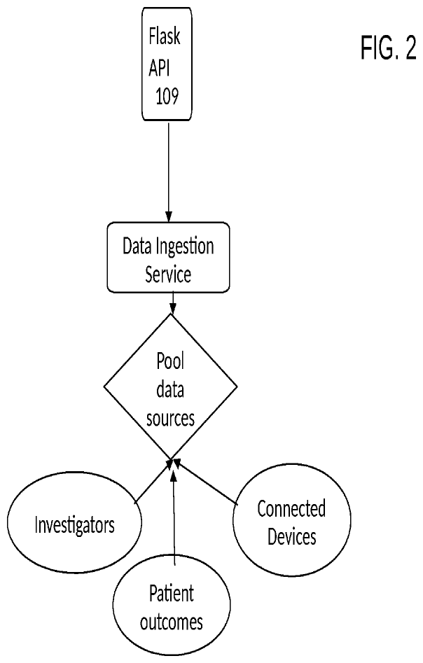

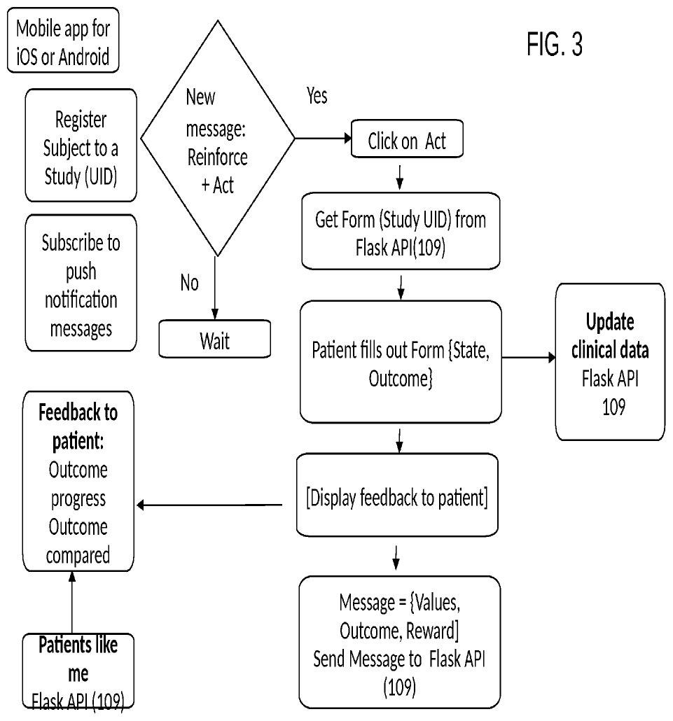

Referring to FIGS. 1A, 1B, 1C, 2, 3 and 7A, 7B and 7C, common entity store 102 is used to host data from a plurality of clinical trial data provider tenants, at least one tenant instance per medical indication, historical clinical trial data study, and data pertaining to a plurality of patients. Control agents 106, training agents 105 and study monitor agents 104 are communicatively coupled to the common entity store 102 through the network 108 to determine a plurality of actions required for the patient compliance based on the historical clinical trial data.

In one implementation, the network 108 may be a wireless network, a wired network or a combination thereof. The network 108 can be implemented as one of the different types of networks, such as an intranet, local area network (LAN), wide area network (WAN), the Internet, and the like. The network 108 may either be a dedicated network or a shared network. The shared network represents an association of the different types of networks that use a variety of protocols, for example, Hypertext Transfer Protocol (HTTP), Transmission Control Protocol/Internet Protocol (TCP/IP), Wireless Application Protocol (WAP), and the like, to communicate with one another. Further, the network 108 may include a variety of network devices, including routers, bridges, servers, computing devices, storage devices, and the like.

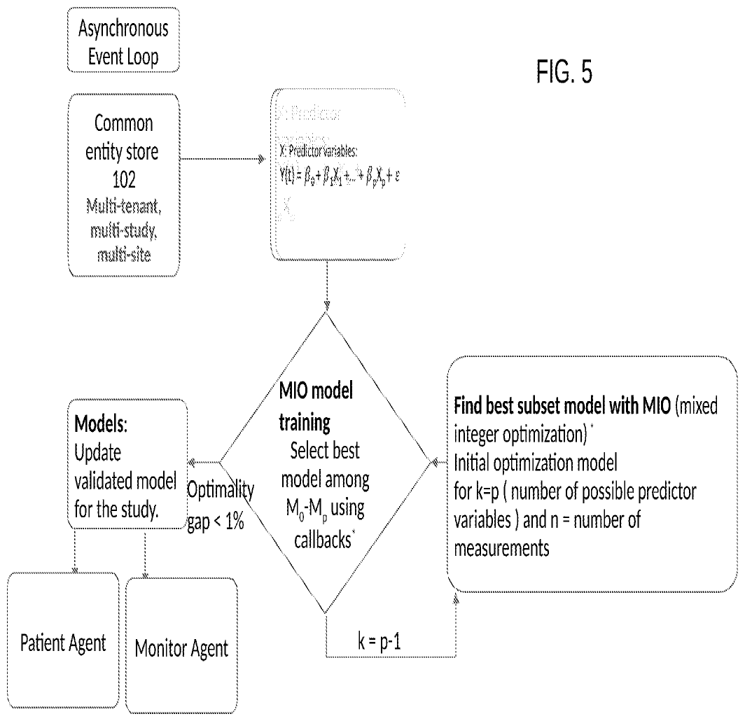

Referring to FIG. 5, training agent 105 learns from examples of non-compliance (and compliance) in historical clinical trial data.

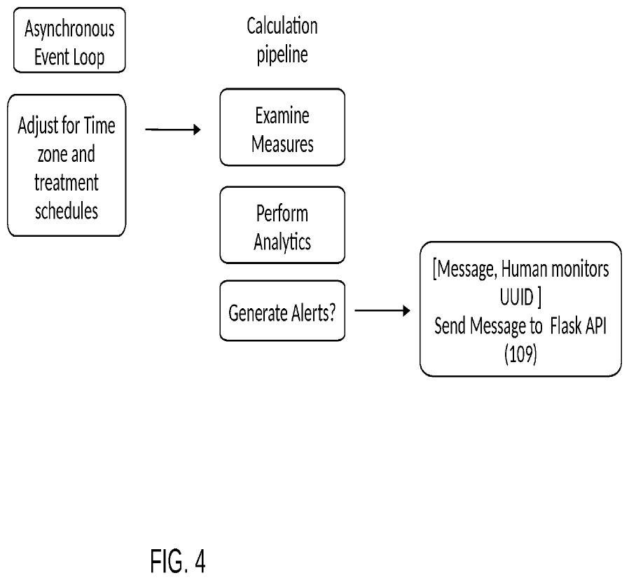

Referring to FIG. 4, study monitor agent 104 enables a human study monitor to define measures and thresholds, receive alerts and graph trends and monitor and improve effectiveness. It automates monitoring of clinical events and measures performance of model. Monitor agent 104 uses clinical measures and analytics. Clinical measures: Aggregate or user-defined functions in PostgreSQL of study data. For example count(rescue meds). We observe that most statistical analytics map well to user-defined aggregates. Analytics: An analytic is a measure function grouped on a clinical trial dimension. For example subject_rescue_meds=count (rescue meds) GROUP BY subject.

Monitor query examples include: Aggregate metrics by patient, location, cohort, study, time period.

Alerts: An alert is triggered when an analytic value is above or below threshold. For example subject_rescue_meds>1

Examples of metrics used by the monitor agent might include:

$$\begin{matrix}Model performance \\ metric \\ {Σ ( {X}_{i} - {Y}_{i} )}^{2} \text{/} T\end{matrix} \begin{matrix}Compliance \\ metric \\ C ( t )\end{matrix} \begin{matrix}CTR \text{-} click \text{-} through rate on \\ message ( Z ) \\ Σ {clicks}^{( Z )} \text{/} Users\end{matrix}$$

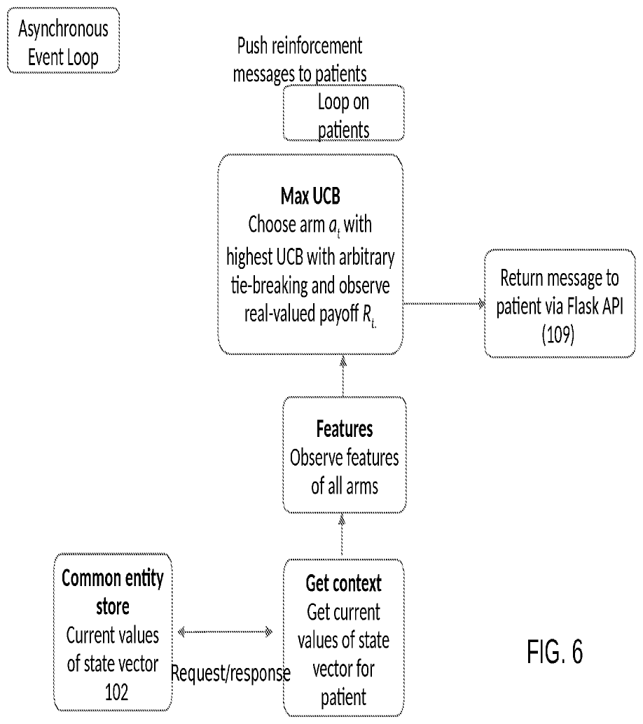

Referring to FIG. 6, control agent 106 uses RL (reinforcement learning) algorithms to provide behavioral reinforcement directly to the patient without relying on physician involvement thereby reducing physician workload and enabling physicians to manage higher-risk exceptions and delegate ongoing compliance to an automated app, caregivers and low-cost phone coaches as applicable.

The patient agent 103 receives reinforcement messages determined by control agent 106 and delivers or displays the reinforcement messages for the study subject or patient. Study monitor agent 104, control agent 106 and patient agent 103 function cooperatively to create a continuous process of monitoring and reinforcement

Referring to FIG. 5, training agent 105 learns from cleaned or validated historical clinical trial data and further creates a model which is composed of a control algorithm+reward function, an expected reward distribution for selecting the plurality of actions from the historical clinical trial data which suggests a highest expected reward. The plurality of actions are functions of measures such as age, gender, and concomitant medical intervention or medication. For instance, a male diabetes patient aged 45 taking aspirin may not be presented with the same suggestions as a female patient aged 25 taking an oral contraceptive.

The model is used by control agent 106. The output of the model is compliance reinforcement messages which are sent by the network connected device configured with control agent 106 via push messaging to the network connected device being used by the subject patient which is configured with patient agent 103 to display, play or otherwise convey the reinforcement message.

Steps for model selection include:

[1] Access historical (and current) clinical data in the common entity store and extract predictor variables:

$$\begin{matrix}X \text{:} Predictor variables \text{:} \\ Y ( t ) = {β}_{a} + {β}_{1} {X}_{1} + … + {β}_{p} {X}_{p} + ɛ\end{matrix}$$

[2] MIO model training: Select best model among M0-Mp using callbacks*

[3] where k=p−1: Find best subset model with MIO (mixed integer optimization) *

Initial optimization model for k=p (number of possible predictor variables) and n=number of measurements.

Further, the patient agent is implemented in the computing unit 103 to directly communicate with the patients and suggest required actions for a given context such as clinical status, previous pattern of responses and social performance of similar patients. The objective of the patient agent is to maximize the expected compliance to the treatment protocol and includes but is not limited to medication-taking, physiotherapy, medical device usage, biological sampling or measurement, etc.

The RL control agent 106 utilizes a reinforcement learning (RL) mechanism to suggest the required actions based on the interaction with the environment (the patient and her devices). In case of the patient compliance, the Patient agent 103 continuously interacts with the patients to propose the actions to the patient and evaluates a continuous reward functions. The continuous reward function incorporates at least three pathways by using the past and current experience of the patients.

Referring to FIG. 6, the RL control agent 106 considers baseline information learned from the historical clinical trial data by the training agent 105 for an expected reward of an action which is a function of a set of measures for a particular indication and patient profile. Second, at a given time duration, the RL control agent 106 refines the expected reward for an action for a particular patient using incremental linear regression of context parameters. Third, the RL control agent 106 estimates and utilizes an upper-confidence-bounds (UCB) for each suggested action in the context to add a “bonus” to the current estimate.

In an embodiment, the patient agent 103 is implemented in a smartphone mobile application such as IOS and Android or as a client-less/server-side solution that communicates with patients using text messaging and a cost-effective dumb phone to make the service accessible to people with limited means and connectivity.

In an embodiment, the present system 100 includes a clinical trial back-end system which is a multi-tenant and a multi-study database. The clinical trial back-end system performs various functions such as sponsor and patient self-service registration 202, ingesting data 204 from clinical trials and medical devices, and generating alerts on measures over the threshold by study monitor agent 104.

In an embodiment, the present system includes a patient back-end system which maintains a multi-tenant and multi-patient database. The functions performed by the patient back-end system includes but not limited to user self-service registration, executing the 3-step algorithm (baseline, refine, bonus) and selecting a message, and sending push notification messages with recommended actions.

In an embodiment, the present system includes a patient front-end system which receives the push notification messages and schedules reminders. Further, the patient front-end system performs a comparative performance analysis with other patients in order to provide tips and tricks while throttling frequency of messages in order to avoid message fatigue where patients are overwhelmed by too many messages and ignore suggestions.

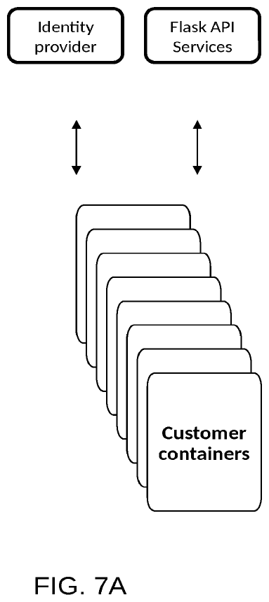

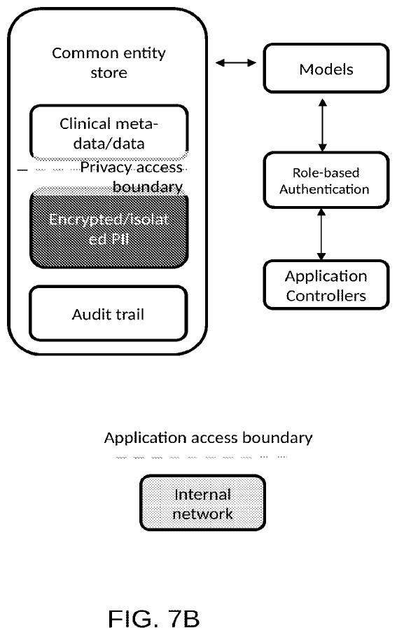

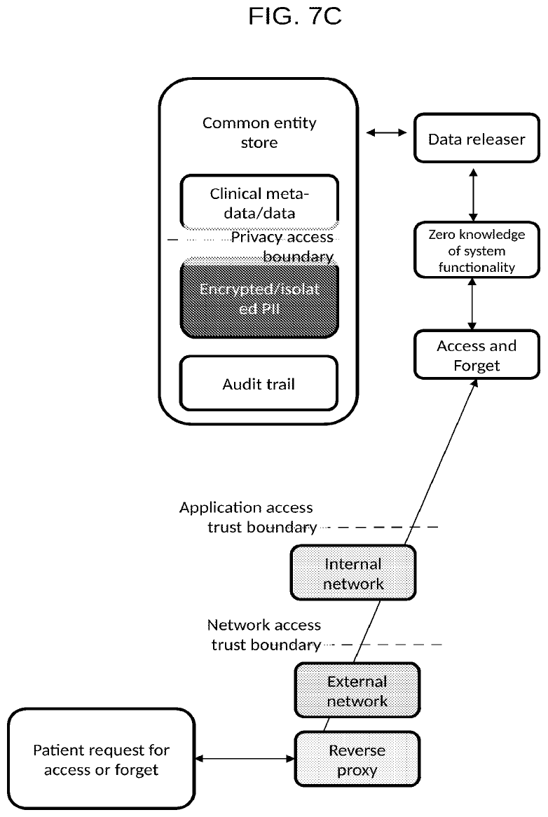

Referring to FIGS. 7A-7C, there are illustrated system features for security and privacy.

Common entity store 102 is optimized for multi-tenants, supporting many customers (study sponsors).

Customer opt-in enables public-access and sharing of models at customer discretion. Customer data is segregated in order to ensure exclusivity of data as well as the compliance models, i.e. the compliance model belongs to the study sponsor.

Personally-identifiable data (PII) from clinical data is isolated. The system 100 identifies a patient for push notification using the device UUID (universal unique identifier) of device, challenge-response question and phone number for recovery if device changes. No other privacy data is needed.

Furthermore, a privacy data release and forget mechanism is provided to enable a subject patient to have his/her data deleted from the common entity store.

FIG. 7A shows Multi-tenancy. Opt-in to share data for open access models

FIG. 7B shows the use of Container: Segregates customers (study sponsors). Isolates personally identifiable information (PII)

FIG. 7C Privacy data access and forget mechanism

A person with ordinary skill in the art will appreciate that the systems, modules, and sub-modules have been illustrated and explained to serve as examples and should not be considered limiting in any manner. It will be further appreciated that the variants of the above-disclosed system elements, modules, and other features and functions, or alternatives thereof, may be combined to create other different systems or applications.

Those skilled in the art will appreciate that any of the aforementioned steps and/or system modules may be suitably replaced, reordered, or removed, and additional steps and/or system modules may be inserted, depending on the needs of a particular application. In addition, the systems of the aforementioned embodiments may be implemented using a wide variety of suitable processes and system modules, and are not limited to any particular computer hardware, software, middleware, firmware, microcode, and the like.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not is limited to the particular embodiment disclosed.

1-16. (canceled)

17. A computing system, comprising:
a processor; and
memory storing instructions that, when executed by the processor, cause the processor to perform acts comprising:
receiving historical clinical trial data for a plurality of patients enrolled in a first clinical trial, wherein the historical clinical trial data comprises characteristics of the plurality of patients when the plurality of patients is enrolled in the first clinical trial;
generating a reward distribution based upon the historical clinical trial data, wherein the reward distribution comprises indications of actions taken by the plurality of patients and values assigned to the actions, wherein the values are associated with compliance of the plurality of patients with restrictions of the first clinical trial;
transmitting a message to a device of a patient that is enrolled in a second clinical trial, wherein the message comprises an indication of an action from the reward distribution that the patient is to perform in order to increase a likelihood of compliance with restrictions of the second clinical trial;
receiving data from the device of the patient, wherein the data is indicative of whether the patient performed the action; and
updating the reward distribution based upon the data received from the device of the patient.

18. The computing system of claim 17, wherein the reward distribution is based upon at least one of:
ages of each of the plurality of patients;
genders of each of the plurality of patients;
medical intervention experienced by each of the plurality of patients; or
medications taken by each of the plurality of patients.

19. The computing system of claim 17, wherein the action is at least one of:
taking a medication;
undergoing physiotherapy;
utilizing a medical device;
taking a biological sample; or
taking a biological measurement.

20. The computing system of claim 17, wherein the device of the patient presents the message to the patient on a display.

21. The computing system of claim 17, wherein the data is a health measurement of the patient, the acts further comprising:
generating an alert when the health measurement exceeds a threshold value.

22. The computing system of claim 17, the acts further comprising:
receiving a clinical status of the patient, wherein the generating the reward distribution is further based upon the clinical status of the patient.

23. The computing system of claim 17, the acts further comprising:
subsequent to generating the reward distribution and prior to transmitting the message, identifying the action from the reward distribution, wherein the action has a highest likelihood of causing the patient to comply with the restrictions of the second clinical trial as compared to remaining actions in the reward distribution.

24. The computing system of claim 17, wherein the device is a smartphone operated by the patient.

25. The computing system of claim 17, wherein the historical clinical trial data is stored in a data store that stores historical clinical trial data for a plurality of sponsors that conduct clinical trials.

26. A method performed by a processor of a computing system, comprising:
receiving historical clinical trial data for a plurality of patients enrolled in an ongoing clinical trial, wherein the historical clinical trial data comprises characteristics of the plurality of patients when the plurality of patients is enrolled in the ongoing clinical trial;
generating a reward distribution based upon the historical clinical trial data, wherein the reward distribution comprises indications of actions taken by the plurality of patients and values assigned to the actions, wherein the values are associated with compliance of the plurality of patients with restrictions of the ongoing clinical trial;
identifying an action in the reward distribution based upon a value assigned to the action;
transmitting a message to a device of a patient that is enrolled in the ongoing clinical trial, wherein the message comprises an indication of an action from the reward distribution that the patient is to perform in order to increase a likelihood of compliance with restrictions of the ongoing clinical trial;
receiving data from the device of the patient, wherein the data is indicative of whether the patient performed the action; and
updating the reward distribution based upon the data received from the device of the patient.

27. The method of claim 26, wherein updating the reward distribution causes the values in the reward distribution to be changed, the method further comprising:
identifying a second action in the reward distribution based upon a second value assigned to the second action, wherein the second value is greater than the value; and
transmitting a second message to the device of the patient that is enrolled in the ongoing clinical trial, wherein the second message comprises an indication of the second action.

28. The method of claim 26, wherein the device of the patient displays the message to the patient based upon a frequency of messages received from the computing system.

29. The method of claim 26, further comprising:
prior to generating the reward distribution, receiving a clinical status of the patient from the device of the patient, wherein the reward distribution is further generated based upon the clinical status of the patient.

30. The method of claim 26, wherein generating the reward distribution comprises:
setting a baseline distribution based upon the historical clinical trial data, the baseline distribution comprising the actions and initial values assigned to the actions;
updating the initial values for each action in the actions based upon an incremental linear regression; and
adding an upper-confidence bound to the updated initial values for each action in the actions.

31. The method of claim 26. wherein the message is a push message that is pushed to the device of the patient.

32. The method of claim 26, wherein the action is at least one of:
taking a medication;
undergoing physiotherapy;
utilizing a medical device;
taking a biological sample; or
taking a biological measurement.

33. The method of claim 26, wherein the reward distribution is based upon at least one of:
ages of each of the plurality of patients;
genders of each of the plurality of patients;
medical intervention experienced by each of the plurality of patients; or
medications taken by each of the plurality of patients.

34. A computer program product comprising instructions that, when executed by a processor, cause the processor to perform acts comprising:
receiving historical clinical trial data for a plurality of patients enrolled in a clinical trial, wherein the historical clinical trial data comprises characteristics of the plurality of patients when the plurality of patients is enrolled in the clinical trial;
generating a reward distribution based upon the historical clinical trial data, wherein the reward distribution comprises indications of actions taken by the plurality of patients and values assigned to the actions, wherein the values are associated with compliance of the plurality of patients with restrictions of the clinical trial;
transmitting a message to a device of a patient that is enrolled in the ongoing clinical trial, wherein the message comprises an indication of an action from the reward distribution that the patient is to perform in order to increase a likelihood of compliance with restrictions of the clinical trial, wherein the device of the patient presents the message to the patient on a display;
receiving data from the device of the patient, wherein the data comprises an indication of whether the patient performed the action; and
updating the reward distribution based upon the data received from the device of the patient.

35. The computer program product of claim 34, wherein the data further comprises a clinical status of the patient.

36. The computer program product of claim 34, the acts further comprising:
subsequent to updating the reward distribution, transmitting a second message to the device of the patient, the second message comprising an indication of a second action from the updated reward distribution.
